(19) United States
(12) Patent Application Publication
(10) Pub. No.: US 2017/0210086 A1
(43) Pub. Date: Jul. 27, 2017

(54) Painless medical adhesive tape easy to be torn

(71) Applicant: CHENG DU XIN AO GUAN MEDICAL EQUIPMENT CO., LTD, Chengdu, Sichuan (CN)

(72) Inventor: Yicheng ZENG, Chengdu, Sichuan (CN)

(21) Appl. No.: 15/328,176

(22) PCT Filed: Dec. 25, 2014
(86) PCT No.: PCT/CN2014/094984

§ 371 Date: Jan. 23, 2017

(30) Foreign Application Priority Data
Jul. 25, 2014 (CN) 201410357867.2

(51) Int. Cl. B32B 3/26; B32B 29/02; B32B 7/12

(52) U.S. Cl. CPC B32B 3/266; B32B 7/12; B32B 29/02; A61F 13/0269

(57) ABSTRACT

A painless medical adhesive tape easy to be torn includes: a layer of upper medical adhesive base fabric, a layer of lower medical adhesive base fabric and a layer of anti-adhesive paper (4), wherein: the layer of lower medical adhesive base fabric (2) has multiple holes (20), and the layer of lower medical adhesive base fabric (2) and the layer of upper medical adhesive base fabric (1) that have a same length and width are combined together; through an adhesive applicator, a medical adhesive (3) is applied to a lower portion of the layer of lower medical adhesive base fabric (2); during an adhesive process, the layer of upper medical adhesive base fabric (1) and the layer of lower medical adhesive base fabric (2) are limitedly adhered together by means of permeation of the medical adhesive (3); and the layer of anti-adhesive paper (4) is directly adhered to the lower portion of the layer of lower medical adhesive base fabric (2) through the medical adhesive (3). Due to the adhesive force between the layer of upper medical adhesive base fabric (1) and the layer of lower medical adhesive base fabric (2) is smaller than the adhesive force between the layer of lower medical adhesive base fabric (2) and the skin, the layer of upper medical adhesive base fabric (1) is firstly removed and then the layer of lower medical adhesive base fabric (2) is torn off, such that through removing the medical adhesive base fabric twice, the tearing pain is able to be remarkably relieved and the comfort level of the patient is able to be improved.

### CROSS REFERENCE OF RELATED APPLICATION

This is a U.S. National Stage under 35 U.S.C 371 of the International Application PCT/CN2014/094984, filed Dec. 25, 2014, which claims priority under 35 U.S.C. 119(a-d) to CN 201410357867.2, filed Jul. 25, 2014.

### BACKGROUND OF THE PRESENT INVENTION

Field of Invention

The present invention relates to an externally medical supply, and more particularly to a painless medical adhesive tape easy to be torn.

Description of Related Arts

Medical tape is a widely used clinical adhesive tape, with the improvement of medical standards, health care workers and patients pay more and more attention to the comfort of medical products. At present, when the hospital used medical tape is peeled off from the people body, the people often feels a burst of “tearing pain”; if the tape is peeled off from the delicate skin of the baby, it is likely to damage the skin, resulting in significant scars.

At present, traditional medical tapes for adults include two layers: a layer of sticky and a layer of non-stick. However, in the neonatal intensive care unit, the traditional tapes are unable to be used again, because once the adhesive tapes are adhered to the joints, it is easy to tear the body skin during the baby's activities, the same damage also occurs in the elderly people with skin fragile. When this situation occurs, in addition to bring psychological and physical pains to patients, medical complaints and disputes may also be caused to have a certain negative impact on the medical unit.

### SUMMARY OF THE PRESENT INVENTION

Aiming at above-mentioned clinical problems of existing medical adhesive tapes, the present invention provides a medical adhesive tape which has simple and reasonable structure and firm adhesion, is easy to be torn and has small pain.

Accordingly, in order to accomplish the above object, the present invention adopts a technical solution as follows.

A painless medical adhesive tape easy to be torn, comprises: a layer of upper medical adhesive base fabric, a layer of lower medical adhesive base fabric and a layer of anti-adhesive paper 4, wherein: the layer of lower medical adhesive base fabric 2 has multiple holes 20, and the layer of lower medical adhesive base fabric 2 and the layer of upper medical adhesive base fabric 1 that have a same length and width are combined together; through an adhesive applicator, a medical adhesive 3 is applied to a lower portion of the layer of lower medical adhesive base fabric 2; during an adhesive process, the layer of upper medical adhesive base fabric 1 and the layer of lower medical adhesive base fabric 2 are limitedly adhered together by means of permeation of the medical adhesive 3; and the layer of anti-adhesive paper 4 is directly adhered to the lower portion of the layer of lower medical adhesive base fabric 2 through the medical adhesive 3.

Furthermore, a total area of the multiple holes 20 is more than one fifth of a total area of the lower medical adhesive base fabric 2.

A design principle of the present invention is as follows: two layers of medical adhesive base fabric are adopted, the layer of lower medical adhesive base fabric has multiple holes, the total area of the multiple holes is more than one fifth of the total area of the lower medical adhesive base fabric, the layer of upper medical adhesive base fabric has the same length and width with the layer of lower medical adhesive base fabric and covers the layer of lower medical adhesive base fabric with the multiple holes, the medical adhesive is applied to one face of the layer of lower medical adhesive base fabric with the multiple holes; and the layer of anti-adhesive paper is adhered to the face of the layer of lower medical adhesive base fabric with the medical adhesive. According to different purposes, the tape is cut into various specifications and models, and then packaged and sterilized to complete production.

Beneficial effects of the present invention are as follows.

The present invention is simple and reasonable in structure, beautiful in appearance and convenient to be used. Before use, one end of the paper packaging bag is firstly torn, the painless medical adhesive tape is drawn out, the anti-adhesive paper is torn off, and the layer of lower medical adhesive base fabric is adhered to body parts needed to be fixed. When the painless medical adhesive tape needs to be torn off, the layer of upper medical adhesive base fabric is firstly removed; now, due to the adhesive force between the layer of upper medical adhesive base fabric and the layer of lower medical adhesive base fabric is smaller than the adhesive force between the layer of lower medical adhesive base fabric and the skin, combined with the fixation of the holes in the layer of lower medical adhesive base fabric, during tearing the layer of upper medical adhesive base fabric, the “pre-tearing” is formed between the layer of lower medical adhesive base fabric and the skin; while tearing off the layer of lower medical adhesive base fabric, due to the action of the holes in the layer of lower medical adhesive base fabric, the pain between the layer of lower medical adhesive base fabric and the skin is significantly reduced, such that through removing the medical adhesive base fabric twice, the tearing pain is able to be remarkably relieved and the comfort level of the patient is able to be improved.

### BRIEF DESCRIPTION OF THE DRAWINGS

FIG. 1 is a structurally schematic view of a painless medical adhesive tape easy to be torn provided by the present invention.

FIG. 2 is a schematic view of a layer of lower medical adhesive base fabric provided by the present invention.

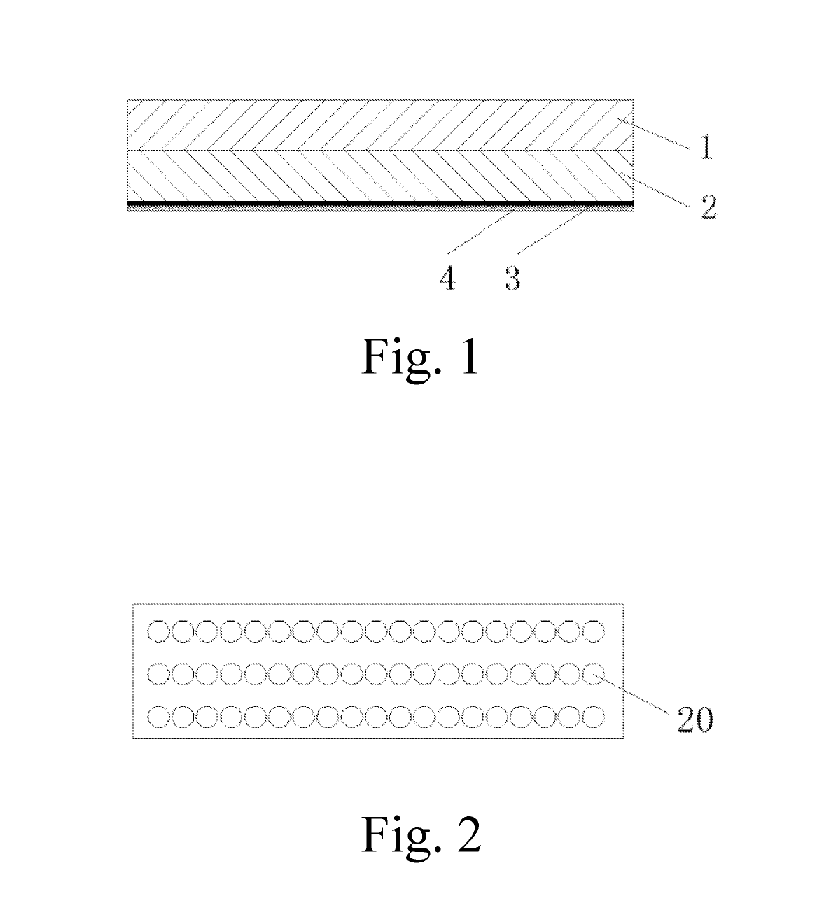

Appended drawing reference signs are: 1 is a layer of upper medical adhesive base fabric; 2 is a layer of lower medical adhesive base fabric; 3 is a medical adhesive; 4 is a layer of anti-adhesive paper 4; and 20 are holes.

### DETAILED DESCRIPTION OF THE PREFERRED EMBODIMENT

The present invention is further explained with drawings as follows.

A painless medical adhesive tape easy to be torn comprises a layer of upper medical adhesive base fabric, a layer of lower medical adhesive base fabric and a layer of anti-adhesive paper 4, wherein: the layer of lower medical adhesive base fabric 2 has multiple holes 20, and the layer of lower medical adhesive base fabric 2 and the layer of upper medical adhesive base fabric that have a same length and width are combined together; through an adhesive applicator, a medical adhesive 3 is applied to a lower portion of the layer of lower medical adhesive base fabric 2; during an adhesive process, the layer of upper medical adhesive base fabric 1 and the layer of lower medical adhesive base fabric 2 are limitedly adhered together by means of permeation of the medical adhesive 3; and the layer of anti-adhesive paper 4 is directly adhered to the lower portion of the layer of lower medical adhesive base fabric 2 through the medical adhesive 3.

While using the painless medical adhesive tape easy to be torn provided by the present invention, one end of a paper packaging bag is firstly torn, the painless medical adhesive tape is drawn out, the anti-adhesive paper 4 is torn off, and the layer of lower medical adhesive base fabric 2 is adhered to body parts needed to be fixed. When the painless medical adhesive tape needs to be torn off, the layer of upper medical adhesive base fabric 1 is firstly removed, and then the layer of lower medical adhesive base fabric 2 which is adhered to human skins is torn off. As a result, through uncovering the medical adhesive tape twice, the tearing pain is able to be remarkably relieved and the comfort level of a patient is able to be improved.

What is claimed is:

1. A painless medical adhesive tape easy to be torn, comprising: a layer of upper medical adhesive base fabric, a layer of lower medical adhesive base fabric and a layer of anti-adhesive paper (4), wherein: the layer of lower medical adhesive base fabric (2) has multiple holes (20), and the layer of lower medical adhesive base fabric (2) and the layer of upper medical adhesive base fabric (1) that have a same length and width are combined together; a medical adhesive (3) is applied to a lower portion of the layer of lower medical adhesive base fabric (2); the layer of upper medical adhesive base fabric (1) and the layer of lower medical adhesive base fabric (2) are limitedly adhered together by means of permeation of the medical adhesive (3); and the layer of anti-adhesive paper (4) is directly adhered to the lower portion of the layer of lower medical adhesive base fabric (2) through the medical adhesive (3).

2. The painless medical adhesive tape easy to be torn, as recited in claim 1, wherein: a total area of the multiple holes (20) is more than one fifth of a total area of the lower medical adhesive base fabric (2).
